(19) United States
(12) Patent Application Publication
(10) Pub. No.: US 2024/0201184 A1
(43) Pub. Date: Jun. 20, 2024

(54) LATERAL FLOW TEST AND LATERAL FLOW TEST SYSTEM

(71) Applicant: Rapid Test Digital Limited, Winchester Hampshire (GB)

(72) Inventor: Nasr-Eddine Djennati, Warrington Cheshire (GB)

(73) Assignee: Rapid Test Digital Limited, Winchester Hampshire (GB)

(21) Appl. No.: 18/555,359

(22) PCT Filed: Apr. 14, 2022
(86) PCT No.: PCT/GB2022/050964

§ 371 Date: Oct. 13, 2023

(30) Foreign Application Priority Data
Apr. 14, 2021 (GB) 2105314.5

(51) Int. Cl. G01N 33/543; G01N 21/84

(52) U.S. Cl. CPC G01N 33/54388; G01N 21/8483

(57) ABSTRACT

A lateral flow test includes a lateral flow assay strip having a sample pad arranged to receive a sample for testing, and a cover operable to move with respect to the strip between a first position in which the sample pad is covered by the cover and a second position in which the sample pad is exposed, and further includes an interlocking element operable to interlock with a lateral flow test reader. The interlocking element is positioned such that the interlocking element interlocking with a lateral flow test reader enables the sample pad to be exposed. Timing for reading the test can be set to start automatically on the test interlocking with a reader, since it is only then that the sample pad can be exposed and that a sample can therefore be deposited. This removes the need for a user to track time or ensure a timer is started.

### TECHNICAL FIELD OF THE INVENTION

The present invention relates to lateral flow tests, lateral flow test systems and methods of conducting a lateral flow test.

### BACKGROUND TO THE INVENTION

Lateral flow assay tests are commonly used for detecting a variety of different analytes, including but not limited to viruses, biomarkers, and/or contaminants. A typical test comprises a strip comprising, in order on the strip, a sample pad, a conjugate pad, and a test area including a test line and a control line. The conjugate pad holds treated detector particles on it which can bind to the analyte to be detected. The test line can bind and hold the analyte on it (and, through holding the analyte, hold the connected detector particle). The control line can bind to and hold the detector particles directly.

In use a sample is applied to the sample pad, which may or may not contain the analyte to be detected. The sample will flow down the strip to the conjugate pad, where the detector particles are removed from the pad and incorporated into the flowing sample. If the analyte is present, detector particles will bind to it. The sample will then flow down to the test area. If the analyte is present it will bind to the test line and be held there, while detector particles not bound to the test line will flow further down the strip and be bound on the control line. The concentration of detector particles darkens or differentially colours the areas in which they collect, forming the test and control lines in the test area. Therefore, the test line becoming visible (or visible enough) demonstrates that the sample contains the analyte. The control line becoming visible demonstrates that the test is operating correctly.

There are two main types of lateral flow assay test: ‘analog’ and ‘digital’ tests. Analog tests show are read by a user viewing the test area by the naked eye and making a subjective determination whether or not a visible test line is present. Digital tests read the test and control lines automatically and output the results to a user, typically on a screen built into the test or by data output to an analytical computer. These results may be qualitative, just like an analog test (positive or negative) and can also produce semi-quantitative results (e.g. high, low or a quantitative number in a range).

Digital tests are easier to use than analog, as they read the test area for a user and eliminate any need for human subjectivity and judgement. However, they are more expensive, which is problematic when a large number of tests need to be carried out (for example, when carrying out mass testing).

One of the difficulties with reading analog tests is that the test must be left for a set period of time before a reading is carried out, to give time for a visible test line to develop. However, it must also be read before too much time has passed, since due to a build up of detector particles at the test line over time a visible line can appear there even if no analyte is present. As such, it is important that a user correctly measures the length of time before reading the test after the sample is deposited on the test, to avoid both false positives and false negatives.

To simplify testing while continuing to keep costs down when carrying out large numbers of tests, a separate reader unit can be used to read the test area and output the results to a user. The reader is set over, or in the case of mobile phone application systems a picture is taken of, the test area when the set time has passed. While this removes subjectivity and judgment of the reading of the test for a user, it still requires them to correctly time from when a sample is deposited on the sample pad and/or start a timer once a sample is deposited.

A lateral flow assay can only be used a single time. A further disadvantage is that over time the test and control lines may fade, so that a used lateral flow assay might be confused with an unused lateral flow assay. This is a reason why a physical indication that an assay has been previously used is desirable.

Embodiments of the present invention seek to overcome/ameliorate these or other disadvantages, and/or provide an improved lateral flow test, lateral flow test system and/or method of carrying out a lateral flow test.

### SUMMARY OF THE INVENTION

According to the first aspect of the present invention there is provided a lateral flow test, the lateral flow test comprising: a lateral flow assay strip comprising a sample pad arranged to receive a sample for testing; and a cover operable to move with respect to the strip between a first position in which the sample pad is covered by the cover and a second position in which the sample pad is exposed; and an interlocking element operable to interlock with a lateral flow test reader, wherein the interlocking element is positioned such that the interlocking element interlocking with a lateral flow test reader enables the sample pad to be exposed.

The advantage of the lateral flow test invention is that a sample can only be deposited once the lateral flow test is inserted in a lateral flow reader since it is only then that the sample pad can be exposed and that a sample can therefore be deposited. Thereby the timing for reading the test can be set to start automatically once the sample is detected after deposition, such deposition only being possible after interlocking with a reader. This removes the need for a user to track the time themselves or ensure a timer is started when the sample is deposited and correctly detected, which makes the system easier to use and reduces the chances of a false positive or negative result being recorded due to incorrect timing.

The interlocking element may be arranged such that, in use, when the interlocking element is interlocked with a lateral flow test reader the cover is moved relative the strip from the first position to the second position. This arrangement means the sample pad is exposed only after interlocking occurs. This ensures that the test is correctly inserted in the reader, and that once a sample is deposited and detected, timing will automatically begin.

The cover may be operable to remain fixed in the second position once it is there. The cover may be operable to remain fixed in the second position once it is there and after removal from interlocking with the reader. This demonstrates the lateral flow assay has been used, so it cannot be visually confused with an unused lateral flow assay and incorrectly used a second time.

The lateral flow test may comprise one or more catches which prevent the lateral flow test moving from the second position to the first position. The catch or catches reduce the chances of a used lateral flow test being mistaken for an unused lateral flow test and being reused.

The catch or one of the catches may extend from the cover. The cover catch may extend from an internal surface of the cover. The cover catch may extend into a space within the cover.

The cover may be lockable in, and unlockable from, the first position. Being lockable in the first position reduces the chances of the sample pad being exposed accidentally.

The interlocking element may be arranged such that, in use, when the interlocking element is interlocked with a lateral flow test reader the cover is unlocked from the first position.

The lateral flow test may comprise a container for containing the lateral flow assay strip. The container may be a cartridge. The cover may form part of the container. The interlocking element may form part of the container. The container may comprise a support on which the lateral flow test strip sits. The support may hold the lateral flow test strip on it.

The support may comprise the catch or one of the catches. The support catch and cover catch may be arranged to, in use, lock with each other to prevent the lateral flow reader moving from the second position to the first position. The support catch may extend from a surface of the support. The support catch may extend into a space within the lateral flow test.

The or each catch may be a tooth. The or each catch may be a saw tooth.

The or each saw tooth may comprise a gentle slope and a steep slope. The steep slope may be perpendicular to a base of the catch. The gentle slope may extend from a base of the catch at an acute angle. The gentle slope may extend to the top of the catch. The horizontal component of the gentle slope may be parallel to the length of the lateral flow test.

The cover catch and support catch may at least partially overlap. The cover catch and support catch may at least partially overlap when looking down the length of the lateral flow test. The cover catch and support catch may partially overlap when looking along the lateral flow test in the direction of motion between the first position and the second position. The gentle slopes of the cover catch and support catch may face each other when the lateral flow test is in the first position. The steep slopes of the cover catch and support catch may face each other when the lateral flow test is in the second position. The gentle slopes may be positioned to slide across each other when the lateral flow test moves from the first position to the second position.

The container may comprise an opening operable to receive a sample for testing through it.

The strip may comprise a test area comprising a test line and a control line. The container may comprise a first aperture through which the test area is visible when the cover is in the second position relative to the strip. The container may comprise a second aperture through which a back of the position at which test area of the lateral flow test strip is positioned is visible when the cover is in the second position relative to the strip. The second aperture may be positioned opposite the first aperture. The second aperture may align with the first aperture. The lateral flow test strip may sit between the first and second apertures.

The cover may be operable to slide relative to the strip to move between the first and second positions.

The cover may comprise an outer body. The strip may be within the cover. The support may sit within the cover. The support and lateral flow test strip may be operable to move together. The lateral flow test strip may reside in a recess within the support.

The support may be positioned relative to the interlocking element such that a reader contacts and pushes the support, therefore moving the cover with respect to the support and strip from the first position to the second position when the interlocking element interlocks with the reader. The support may be positioned relative to the interlocking element such that a reader contacts and pushes the support, therefore moving the cover with respect to the support and strip from the first position to the second position as the interlocking element interlocks with the reader.

The cover may be a hollow cylinder. The cylinder may have a stadium cross section.

The interlocking element may form part of the cover. The interlocking element may comprise an entrance for receiving a part of a reader. The support may be positioned adjacent the entrance. The entrance may be in a second end of the cover. The entrance may be sized such that the support may not move into or through it.

The cover may comprise the opening. The opening may be positioned such that when the cover is in the second position it is aligned with the sample pad. The opening may be positioned such that when the cover is in the first position it is out of alignment with the sample pad. The support may be shaped such that when the cover is in the first position the support is between it and the sample pad. The recess and the opening may be positioned to align when the cover is in the second position. An end of the recess and the opening may be positioned to align when the cover is in the second position. The opening may be positioned to be over the recess when the cover is in the second position. The opening may be positioned to be over an end of the recess when the cover is in the second position.

The cover may comprise the first aperture and/or second aperture. The support may comprise a third aperture operable to align with the second aperture when the cover is in the second position. The first aperture may align with the recess of the support.

The support may slide within the cover to move the cover between the first and second positions. The cover may comprise an outlet through which the support may extend when the cover is in the second position. The outlet may be positioned opposite the entrance in the cover. The outlet may be in a first end of the cover. The support may be positioned to be entirely within the cover when the cover is in the first position.

The support may be an outer body. The strip may be within the support. The cover may sit within the support. The cover may slide within the support to move the cover between the first and second positions.

The support may comprise the opening. The cover may comprise a passageway for receiving a sample to be tested. The passageway may be positioned such that when the cover is in the first position it is out of alignment with the opening and/or sample pad. The passageway may be positioned such that when the cover is in the second position it is aligned with the opening and/or the sample pad.

The support may comprise an outlet through which the cover may extend when the cover is in the first position. The outlet may be in a second short face of the support. The cover may be positioned to be entirely within the support when the cover is in the second position.

The interlocking element may comprise one or more slots in the support, the or each slot operable to receive part of the cover when the cover is in the first position. The cover may be operable to move between the first and second positions in a first direction. The or each slot may extend through the support is a second direction which is non-parallel to the first direction. The second direction may be perpendicular to the first direction. The part of the cover may be a foot or feet of the cover. The cover may comprise a leg or legs connecting the foot or feet to the rest of the cover. The cover may be arranged such that the part of the cover is moved out of the or each slot when interlocking occurs. The part of the cover may be moved within the support.

The foot or feet may form the cover catch. Each foot may be a saw tooth.

According to a second aspect of the present invention there is provided a lateral flow test system comprising a lateral flow test according to the first aspect and a lateral flow test reader operable to read the results of the lateral flow test and output a reading, the lateral flow test reader comprising a counterpart interlocking element, the counterpart interlocking element being the counterpart to the interlocking element of the lateral flow assay test and being operable to interlock with the interlocking element of the lateral flow assay test.

The second aspect of the present invention may comprise any or all of the optional features of the first aspect, as desired or appropriate.

The reader may be operable to accept insertion of the test through it. The reader may comprise an opening sized and shaped to accept the lateral flow test. The opening and container may be sized and shaped to form an interference fit. The opening may be in a surface of the reader.

The reader may comprise one or more shelves operable to support the lateral flow test when interlocked. The shelves may extend within the reader.

The reader may comprise a passageway sized and shaped to accept the lateral flow test when inserted into the reader. The passageway may extend within the lateral flow test reader. The opening may form the entrance of the passageway. The or each shelf may form the base of the passageway. An internal wall of the lateral flow test reader may form an end of the passageway. The counterpart interlocking element may be on the on the internal wall at the end of the passageway.

The lateral flow reader may be operable to output a digital reading of the test results of the lateral flow test. The lateral flow reader may comprise a screen operable to output the digital read. The screen may be a touchscreen. The lateral flow reader may be operable to output a digital reading of the test results of the lateral flow test to a database.

The lateral flow reader may comprise one or more line detectors positioned and operable to read the test area of the strip when the lateral flow test is interlocked. The or each line detector may be operable to detect light transmitted or blocked from a respective position on the strip. The or each line detector may be positioned to face the first aperture when the lateral flow test is interlocked.

A first line detector may be positioned to detect the light from or blocked by a control line of the strip when the lateral flow test is interlocked. A second line detector may be positioned to detect light from or blocked by a position on the strip adjacent the control line when the lateral flow test is interlocked. In use, it may be the case that a user does not interlock the lateral flow test perfectly with the reader. They may interlock enough to enable the sample pad to be exposed, but not enough for the lateral flow test to be in the correct position relative the reader. In such situations, the first line detector may not measure light from the control line. Having the second line detector positioned to detect light from an adjacent position means the second line detector may instead measure light from the control line when the lateral flow test and lateral flow test reader are not perfectly interlocked. As such, this feature means the lateral flow test reader may still read the control line even when the lateral flow test and lateral flow test reader are not perfectly interlocked.

The second line detector may be positioned further from the counterpart interlocking element than the first line detector. An imperfect interlock typically means the lateral flow test is not far enough onto or into the counterpart interlocking element. As such, for an imperfect interlock the test line will be positioned further from the counterpart interlocking element than when the interlock is perfect.

A third line detector may be positioned to measure light from a test line when the lateral flow test is interlocked. A fourth line detector may be positioned to measure light from a position on the strip adjacent the test line when the lateral flow test is interlocked.

The fourth line detector may be positioned further from the counterpart interlocking element than the third line detector.

The lateral flow test reader may be operable to read the control line based on the difference between outputs of the first and second detector. The lateral flow test reader may be operable to read the test line based on the difference between outputs of the third and fourth detector. By reading based on the differences, it does not matter which of each pair of detectors detects light from the respective line.

The lateral flow test reader may be operable to start a first timer for a first set period, after which it reads the test lines of the strip. The lateral flow test reader may be operable to detect when the lateral flow test is interlocked. The lateral flow test reader may comprise an interlocking detector operable to detect when interlocking occurs. The lateral flow test reader may be operable to start the first timer when interlocking is detected and when a sample is correctly deposited. The interlocking detector may comprise a pressure detector.

The lateral flow test reader may be operable to start a second timer for a second set period, after which it reads the test lines of the strip. The lateral flow test reader may be operable to start the second timer after the or each line detector outputs an increased reading. By running a second timer from when the line detectors output an increased reading, the reader runs a timer from when the sample reaches the test area (since when the sample reaches the test area the strip will be rendered more translucent, and so more light will be transmitted to the line detectors). The first and second set periods may be equal.

The reader may comprise a light source operable to direct light to the strip when the lateral flow test is interlocked. The light source may be positioned to direct light to the test lines of the strip when the lateral flow test is interlocked. The light source may be positioned to direct light through the second aperture of the lateral flow test when it is interlocked. The light source may be positioned opposite the detectors.

The cooperating interlocking element may comprise a projection sized and shaped to fit into the entrance of the lateral flow assay test. The projection and entrance may be sized and shaped to form an interference fit when interlocked. The projection may be sized and shaped to extend through the entrance, extending within the cover, when interlocked. The projection may extend from a surface of the reader. The projection may extend from an internal surface of the reader, extending within the reader.

The opening may be positioned in the reader opposite the projection, wherein the lateral flow test extends between the opening and the projection when interlocked. The projection may be positioned at the end of the passageway.

The counterpart interlocking element may comprise one or more projections, the or each projection sized and shaped to correspond to a respective slot. When there is a plurality of projections and slots, the projections may be positioned to interlock with the respective slots at the same time. The or each projection and the respective slot may form an interference fit. The or each projection may be retractable from and extendable into the passageway.

The or each projection may be positioned within the passageway. The or each projection may be displaceable from their original position. The or each projection may be biased towards their original position. The reader may comprise one or more biasing means to bias the or each projection to their original positions.

According to a third aspect of the present invention there is provided a method of performing a lateral flow test, comprising the steps of:
(a) providing a lateral flow test system according to the second aspect;
(b) interlocking the lateral flow test and the lateral flow test reader once interlocking has occurred;
(c) the lateral flow test reader automatically starting a timer for a set period; and
(d) the lateral flow test reader reading the test results after the set period.

The advantage of this method is that the timing for reading the test can be set to start automatically on the test interlocking with a reader and a sample being deposited, since it is only then that the sample pad can be exposed and that a sample can therefore be deposited. This removes the need for a user to track the time themselves or ensure a timer is started when the sample is deposited, which makes the system easier to use and reduces the chances of a false positive or negative result being recorded due to incorrect timing.

The method of the third aspect may have any or all of the optional features of the second aspect, as desired or appropriate.

### DETAILED DESCRIPTION OF THE INVENTION

In order that the invention may be more clearly understood one or more embodiments thereof will now be described, by way of example only, with reference to the accompanying drawings, of which:

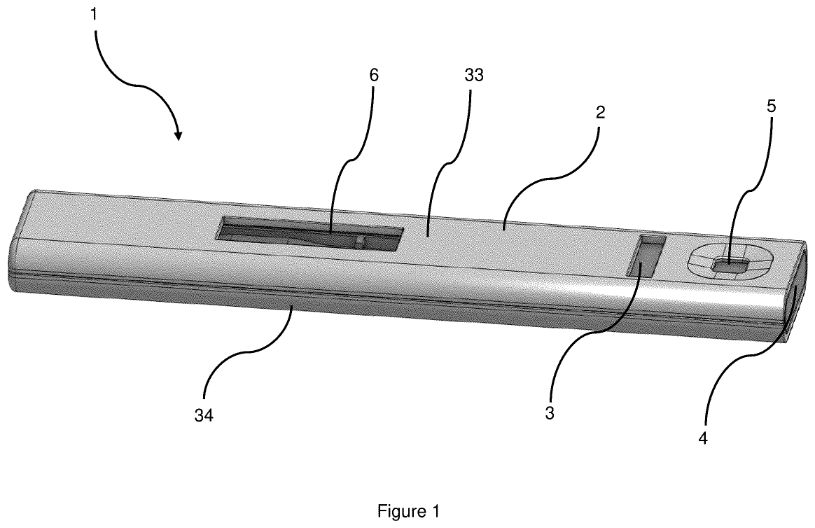

FIG. 1 is a perspective view of a lateral flow test;

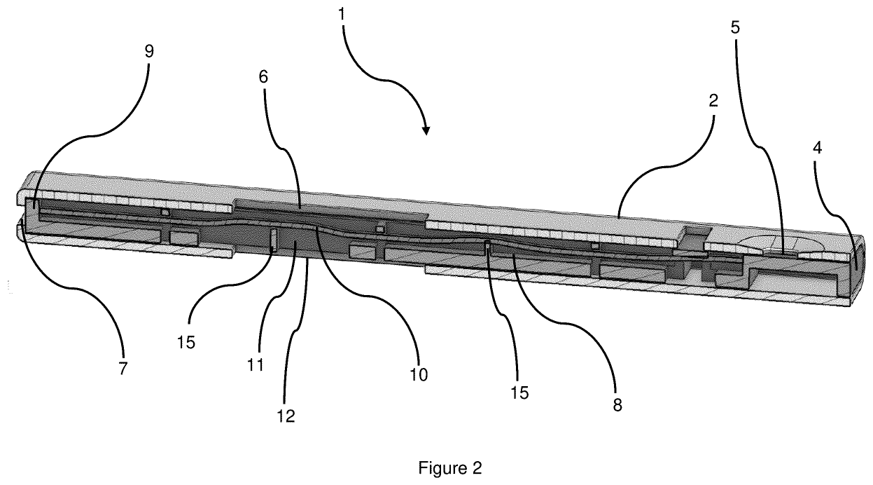

FIG. 2 is a perspective view of a vertical cross-section of the lateral flow test of FIG. 1;

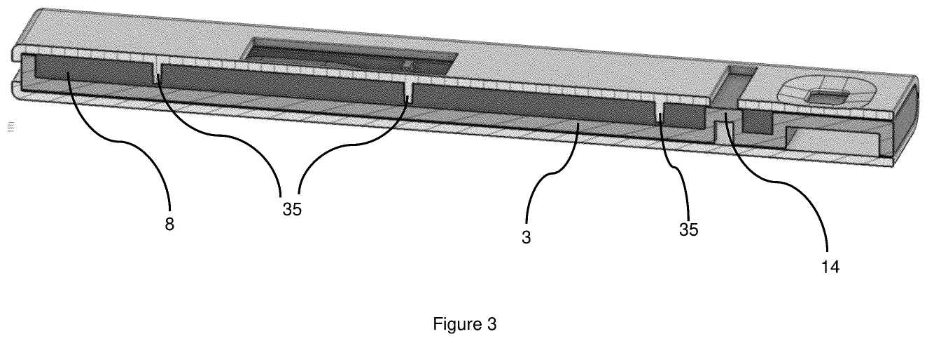

FIG. 3 is a perspective view of an off-centre vertical cross-section of the lateral flow test of FIG. 1;

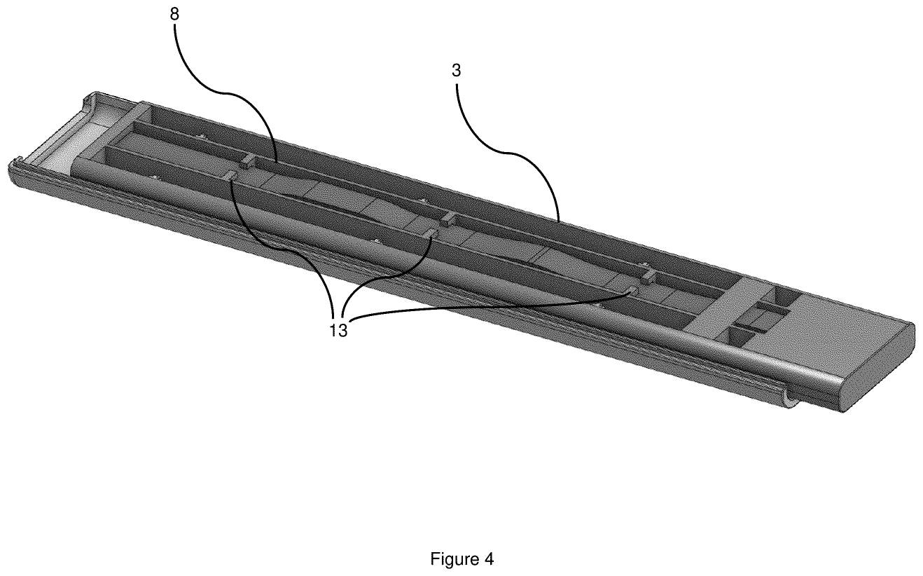

FIG. 4 is a cut-away view of the lateral flow test of FIG. 1;

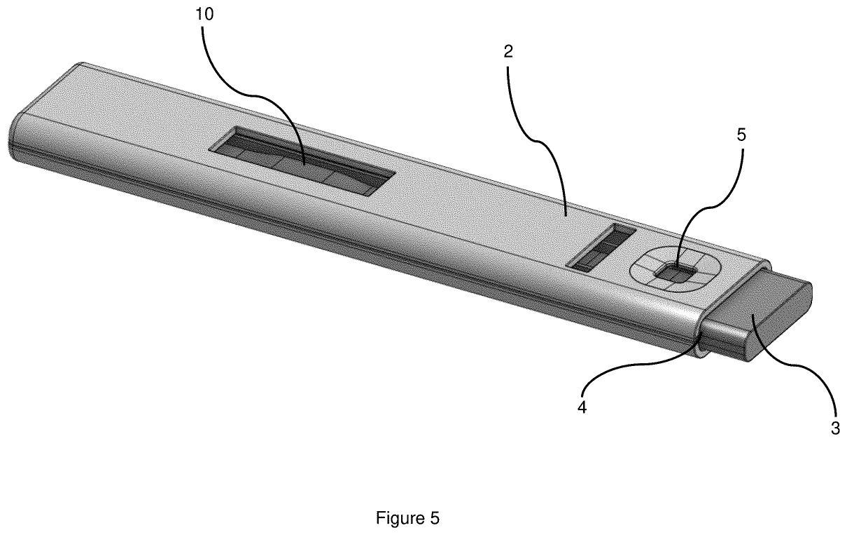

FIG. 5 is a second perspective view of a lateral flow test;

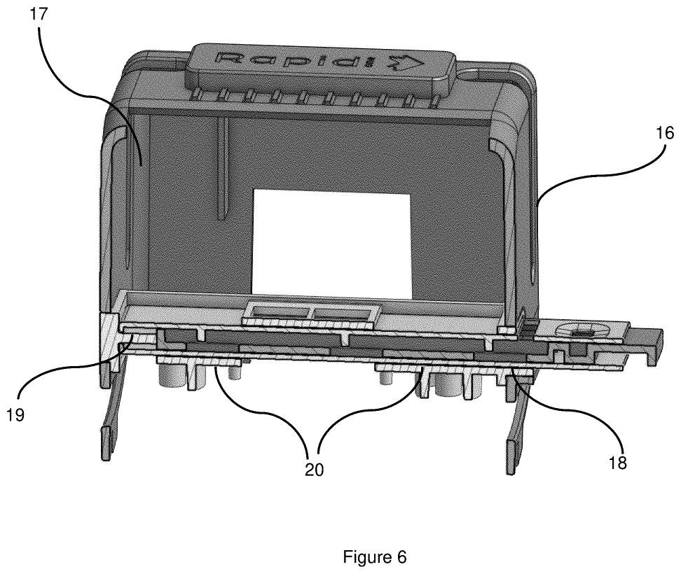

FIG. 6 is a perspective view of a vertical cross-section of a lateral flow reader and the lateral flow test of FIG. 1, which are interlocked;

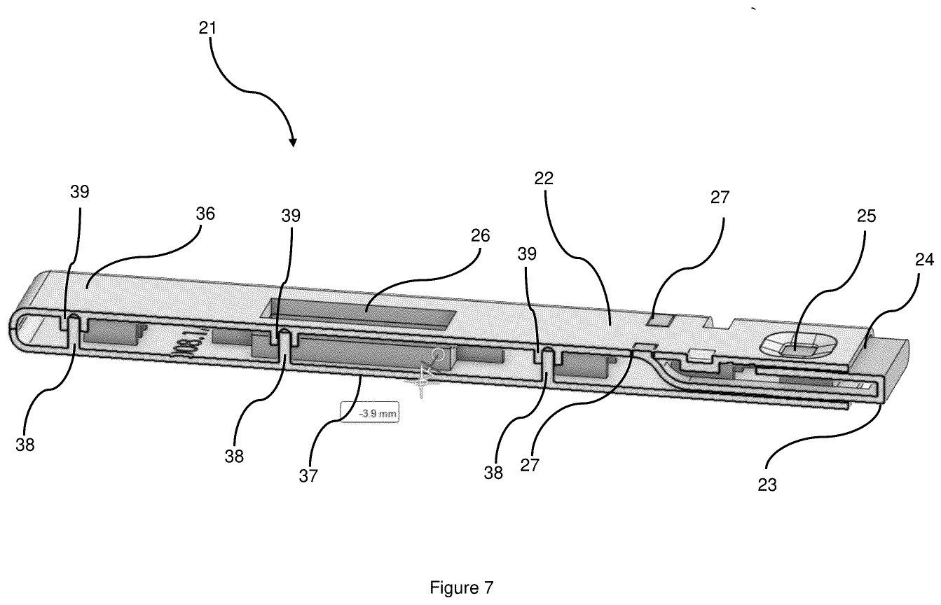

FIG. 7 is a perspective view of an off-centre vertical cross-section of a lateral flow test;

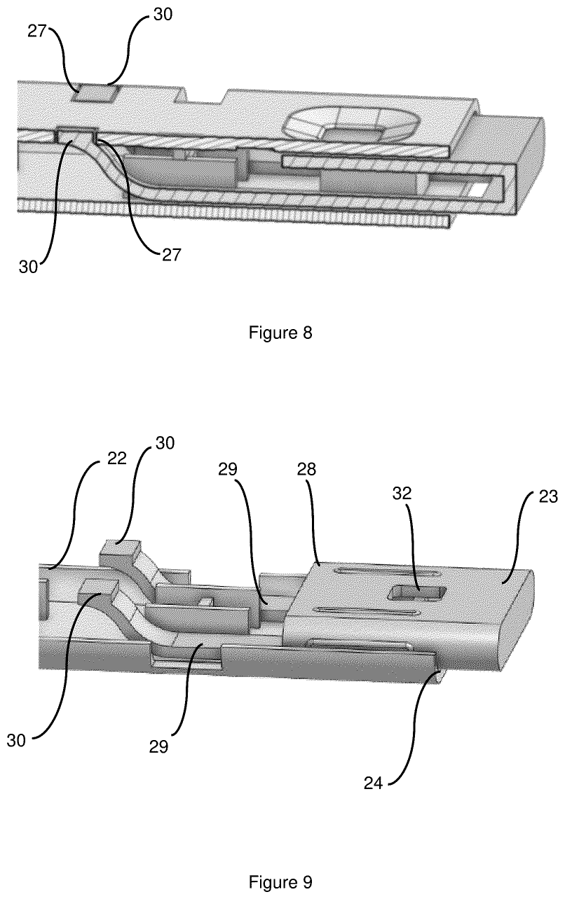

FIG. 8 is a close-up perspective view of an off-centre vertical cross-section of part of the lateral flow test of FIG. 7;

FIG. 9 is a close-up of a cut-away of part of the lateral flow test of FIG. 7;

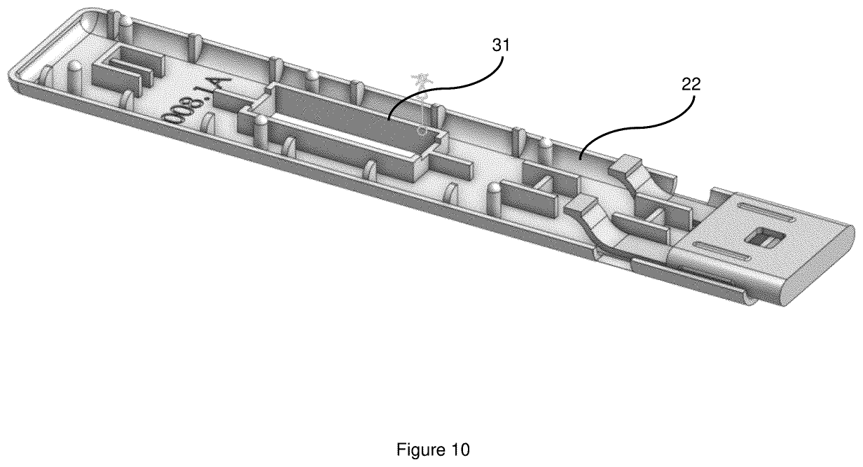

FIG. 10 is a cut-away of the lateral flow test of FIG. 7;

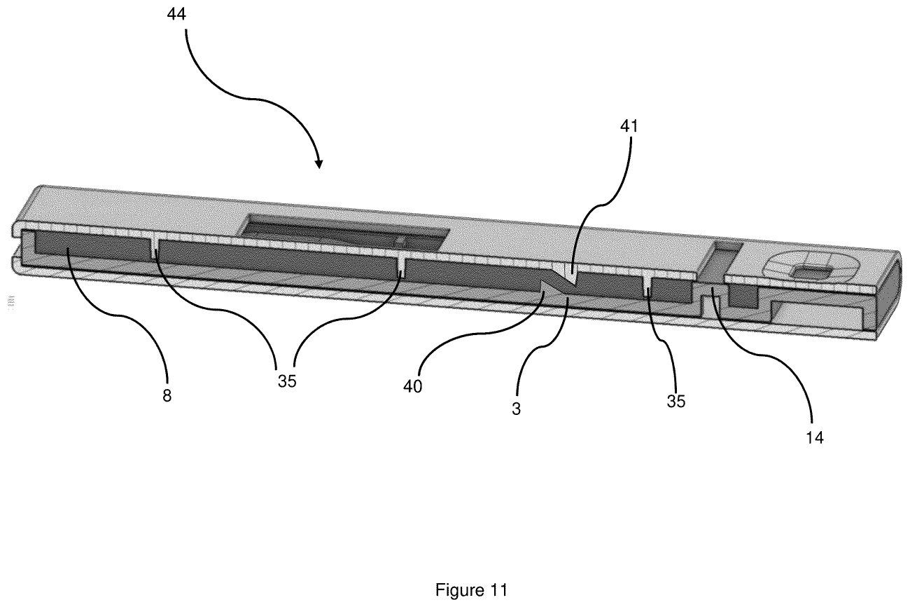

FIG. 11 is a perspective view of an off-centre vertical cross-section of a lateral flow test;

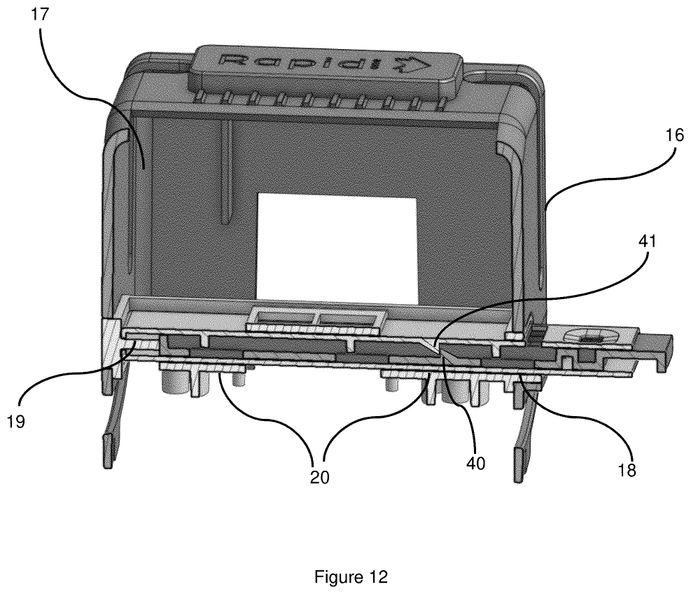

FIG. 12 is a perspective view of a vertical cross-section of a lateral flow reader and the lateral flow test of FIG. 11, which are interlocked;

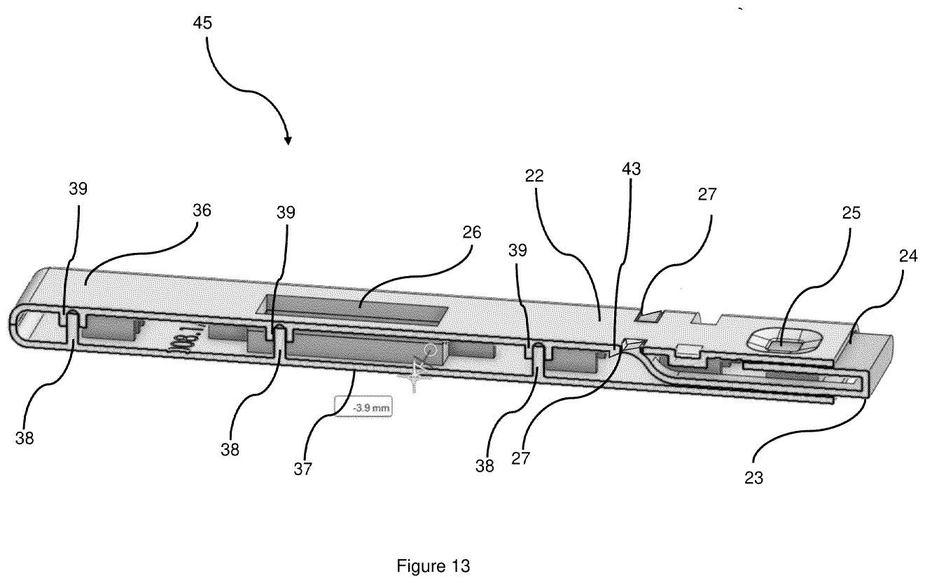

FIG. 13 is a perspective view of an off-centre vertical cross-section of a lateral flow test;

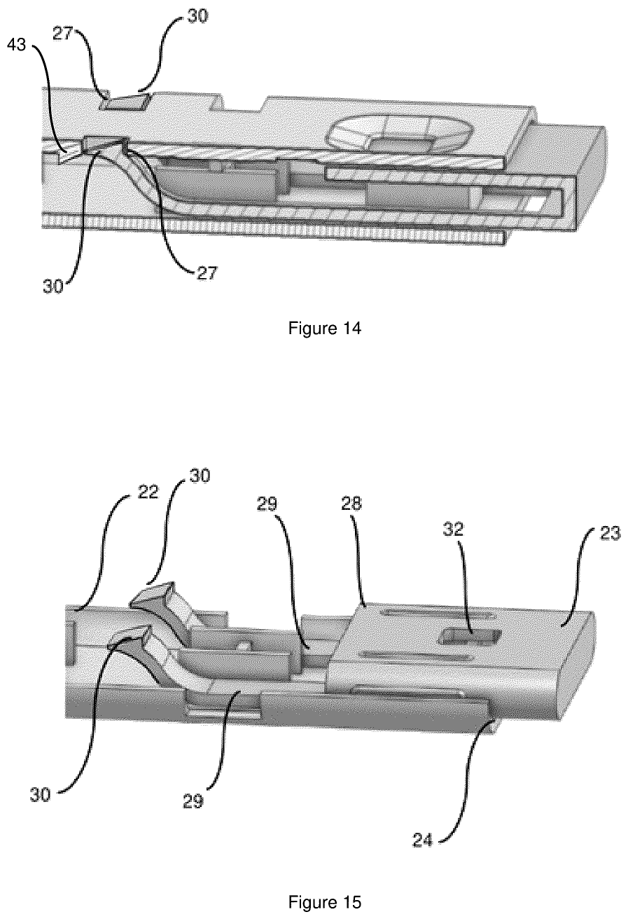

FIG. 14 is a close-up perspective view of an off-centre vertical cross-section of part of the lateral flow test of FIG. 13;

FIG. 15 is a close-up of a cut-away of part of the lateral flow test of FIG. 13; and

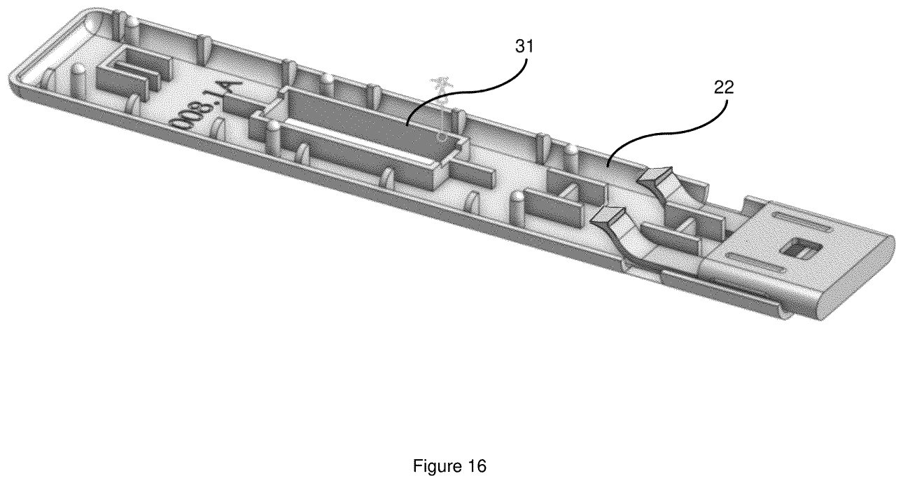

FIG. 16 is a cut-away of the lateral flow test of FIG. 13.

As shown in FIG. 1, a first embodiment of a lateral flow test 1 comprises a cartridge formed by a cover 2 and a support 3. The cover 2 forms the main body of the cartridge. The cover 2 is a hollow cylinder which has a stadium short vertical cross-section. A first end of the cover 2 is open, forming an outlet 4. The cover 2 also comprises an opening 5 in a first long rectangular side. The opening 5 has a rounded square shape. It is situated near the first end of the cover 2, in a slight depression in the surface of the cover 2.

The cover 2 also comprises a first aperture 6 in the first long rectangular side. The first aperture 6 is rectangular, and positioned with one end adjacent the centre of the cover 2 and extending to around halfway to the second end of the cover 2.

The support 3 sits within the cover 2, and when in the first position shown in FIG. 1 is entirely within the cover 2.

As shown in FIG. 2, the cover 2 comprises an entrance 7. The entrance is in the second end of the cover 2. The entrance 7 and support 3 are sized such that the support 3 cannot move through the entrance 7.

The body of the cover 2 is comprised of two panels, a top panel 33 and a bottom panel 34. The body splits down the middle horizontally, with the top half formed by the top panel 33 and the bottom half formed by the bottom panel 34. As shown most clearly in FIG. 3, the top panel 33 comprises a set of first cooperating parts in the form of shafts 35 which extend from the internal surface of the top panel 33 towards the bottom panel 34.

The bottom panel 34 comprises a set of second cooperating parts in the form of pedestals (not shown). When forming the test 1, each shaft 35 slots into a respective pedestal, forming an interference fit and holding the top and bottom panels 33, 34 together.

The support 3 is a cylindrical body with a stadium short vertical cross-section, sized to fit snugly within the cover 2. The support 3 has a recess 8 within a first long rectangular side. The first long rectangular side of the support 3 corresponding to the long rectangular side of the cover 2. The recess 8 extends from a distance from a distance from a first end of the support 3 to a wall 9 at the second end of the support 3, the first and second ends of the support 3 corresponding to the first and second ends of the cover 2. When the cover is in the first position as shown in FIG. 2 the wall 9 is adjacent the entrance 7 of the cover 2, while the first end of the support 3 sits within the outlet 4.

A lateral flow test strip 10 sits within the recess 8, extending along the entirety of the length of the recess 8 and contacting both ends. The strip 10 has a sample pad at one end, and a test area partway along its length comprising a control line and a test line, and a conjugate pad between the sample pad and the test area. The conjugate pad has detection particles which can connect to the appropriate analyte.

The cover 2 has a second aperture 12, in its second long rectangular side, the second aperture 12 is the same size and shape as the first aperture 6, and is positioned in line with it.

The support 3 has a third aperture 11 in the base of the recess 8. The third aperture 11 sits between the centre of the support 3 and the second end of the recess 8, nearer the centre than the second end.

When the cover 2 is in the first position with respect to the strip 10 and the support 3, as shown in FIG. 2, the third and second apertures 11, 12 overlap but are not aligned. The opening 5 of the cover opens onto the surface of the support 3.

As most clearly seen in FIG. 4, the support 3 comprises three pairs of clips 13 within the recess 8. Each clip is a right-angled bar which is adjacent to a side of the recess, a first leg of the bar extending along the internal surface of the side and a second leg extending across part of the top of the recess. Each clip of a pair of clips 13 are positioned on opposite sides of the recess, their second legs extending towards each other. The second legs of each pair of clips 13 leave a space between them. One paid of clips 13 is near the middle of the recess, another is near the first end of the support 3 and a third is near the second end of the support 3.

As shown most clearly in FIG. 3, the recess 8 of support 3 is split into two by a panel 14 across the top of the recess 8. The panel 14 is positioned near the end of the recess 8 towards the first end of the support 3.

As shown most clearly in FIG. 2, there are struts 15 within the recess 8. Each strut 15 extends from one side of the recess 8 to the other, and extends from the bottom of the recess 8 to roughly halfway up. One strut 15 is in the middle of the third aperture 11, while the other strut 15 is near the middle of the support 3.

In use, the strip 10 is placed within the recess 8 of the support, being bent through the pairs of clips 13 before being allowed to resume its original shape. The pairs of clips 13 then hold the strip 10 within the recess 8. The strip sits on the struts 15. The strip 10 is also thread beneath the panel 14 such that it sits within both parts of the recess 8, the sample pad siting in the smaller part of the recess 8 near the first end of the support 3.

As shown most clearly in FIG. 5, the cover 2 and support 3 can move relative to each other to a second position. The support 3 moves partially out of the outlet 4, until the sample pad of the strip 10 and the opening 5 are aligned, the opening 5 opening onto the sample pad. In the second position the second and third apertures 12, 11 are also aligned.

FIG. 6 shows a reader 16 comprising a relatively cuboidal body 17, but with one corner chamfered off to form a diagonal surface. The reader 16 comprises an opening 18 in the cuboidal body 17, opening into the hollow internal area of the cuboidal body 17. Within this area, on a surface opposite the opening 18, is a projection 19. The opening 18 is sized and shaped to accept the test 1, and the projection 19 is sized and shaped to fit into the entrance 7 of the test 1. Between the projection 19 and the entrance 7 is a passageway formed by the hollow internal area of the cuboidal body 17.

The reader 16 comprises two of shelves 20, extending between sides of the cuboidal body 17 and across the hollow internal area, extending perpendicularly to the passageway. The shelves form the base of the passageway. One shelf 20 is formed together with the opening 18, and extends for a distance out of the opening 18. The other shelf 20 is next to the projection 19. Both shelves 20 are positioned such that they do not cover the second aperture 12 of the test 1 when the test is within the reader 16.

In use the test 1 is inserted through the opening 18 of the reader 16 and down the passageway, supported on the shelves 20. Test 1 is pushed along the passageway until it contacts the wall of the internal area opposite the opening 18. As this occurs, the projection 19 interlocks by entering the entrance 7 of the test 1. The projection 19 pushes the support 3 away from the entrance 7, thereby moving the cover 2 and support 3 from the first position to the second position. As such, the sample pad is only exposed such that a sample for testing can be deposited on it when the test 1 is fully inserted into the reader 16.

The reader 16 comprises a pressure sensor (not shown) positioned adjacent the projection 19 on the internal wall of the reader 16. In use this pressure sensor detects when the test 1 contacts it, and so detects when the entrance 7 and projection 19 are interlocked. The reader 16 is operable to start a timer when the pressure sensor outputs that contact is detected.

The reader 16 also comprises four light detectors (not shown) which are positioned on the ceiling of the internal area of the reader 16 such that they are opposite to and facing the first aperture 6 of the test 1 when the test is interlocked with the projection 19. Specifically, a first light detector is positioned so as to be opposite the control line of the strip 10, a second light detector is positioned so as to be opposite a point on the strip 10 adjacent the control line, a third light detector is positioned so as to be opposite the test line of the strip 10, and the fourth light detector is positioned so as to be opposite a point on the strip 10 adjacent the test line. The reader 16 is operable to read the differential between the control pair of detectors, the first and second detectors, and the differential between the test pair of detectors, the third and fourth detectors.

The reader 16 also comprises a light source (not shown), which is positioned on the floor of the internal area of the reader 16 to be opposite to and facing the second aperture 12 of the test 1 when the test is interlocked with the projection 19. The reader 16 is operable to turn on the light source when interlocking is detected by the pressure sensor.

In use, a test 1 is inserted into the reader 16 and pushed through the passageway until the projection 19 and entrance 7 are interlocked. The pressure sensor detects the interlocking, and the reader 16 starts the first timer and turns on the light source.

Once the test 1 and reader 16 are interlocked, the user will be able to deposit a sample. The sample will flow along the strip 10 from the sample pad to the control line and test line. The presence of the sample on the strip 10 renders it more translucent. As such, as the sample reaches the test area of the strip 10 more light from the light source reaches the light detectors. Given the sample travels along the strip 10, more light will reach one light detector of each pair before the other light detector, resulting in a differential signal outputting from the pair of detectors until both detectors receive the increased amount of light. The reader 16 begins a second timer after differential signals from both pairs of detectors have been detected and are no longer being outputted.

The first and second timers each run for the same set period, this set period being the recommended amount of time to wait until reading the test area of the strip 10. Once each timer stops, the reader 16 checks the outputs of each pair of detectors, a differential signal above a set threshold for the control pair of detectors results in the reader 16 outputting that the test 1 is operating correctly, while no differential signal (or a differential signal below a set threshold) for the control pair of detectors results in the reader 16 outputting that the test 1 is void.

If the test 1 is operating correctly, the reader 16 also outputs the results from the test pair of detectors. A differential signal above a set threshold means the sample does contain the analytes the test 1 is looking for, while no differential signal (or a differential signal below a set threshold) means the samples does not contain the analytes.

Once a test 1 has been read by the reader 16, it can be removed from the reader 16 and disposed of. A new test 1 can then be inserted into the reader 16 for further testing.

The reader 16 also comprises a touchscreen (not shown), via which it can output the results. A user can also set the timer periods and/or the differential signal thresholds via this touchscreen.

The reader comprises a memory (not shown), which can store the thresholds and timer periods for various different types of tests 1 (for various different analytes). A user can then select the type of test 1 which is to be used via the touchscreen, and the reader 16 will automatically change the timer periods and thresholds to those given in the memory. The reader 16 will also change the wording of the output to reflect the type of test 1, based on information in the memory.

FIG. 7 shows a second embodiment of a lateral flow assay test 21. The test 21 comprises a cartridge formed by a support 22 and a cover 23. The support 22 is the main body of the cartridge. The support 22 is a relatively rectangular, hollow body, but which is a half stadium vertical long cross section at a second end. At the first end the support 22 is open to form an outlet 24.

The support 22 has an opening 25 near the first end, situated in the centre of a depression on the long side of the body, and a first aperture 26 near the centre of the body on the long side, lying between the centre and the second end. The support also has two slots 27 formed in the long side, situated between the opening 25 and the first aperture 26.

As shown in FIG. 10, the support 22 also comprises a second aperture 31 on the bottom, second long side. The second aperture 31 is aligned with the first aperture 26.

As shown most clearly in FIG. 7, the body of the support 22 is formed of a top panel 36 and a bottom panel 37. The body is split along the horizontal cross-section, the top half being the top panel 36 and the bottom half being the bottom panel 37. The bottom panel 37 comprises a set of cooperating pats in the form of shafts 38 which extend from the internal surface of the bottom panel 37 towards the top panel 36.

The top panel 36 comprises a set of second cooperating parts in the form of pedestals 39. When forming the test 21, each shaft 38 slots into a respective pedestal 39, forming an interference fit and holding the top and bottom panels 36, 37 together.

The cover 23 is situated within the support 22, at the first end. In the first position shown in FIG. 7, the cover 23 extends partially out of the outlet 24. The cover 23 is operable to slide within and relative to the support 22, up and down the support 22.

As most clearly seen in FIG. 9, the cover 23 comprises a body 28 which is a cylinder with a stadium cross-section. The body 28 is sized to fit snugly within the support 22, In the first position shown in FIG. 9 a first end of the body 28 sits outside the support 22, roughly a third of the body 28 extending out of the outlet 24. The other two thirds of the body 28 lie within the support 22, including the second end. The body 28 is hollow, and open at the second end.

Two legs 29 extend from the second end of the body 28, further into the support 22. Each leg 29 extends from the bottom of the second end of the body 28, and each is adjacent a respective curved side of the body 28. The legs 29 each extend for a length perpendicular from the second end, before curving up towards the top long side of the support 22. Each leg 29 ends in a respective foot 30. Each foot 30 is a pad with a square cross-section. As most clearly shown in FIG. 8, in the first position the feet 30 sit within the slots 27. With the feet 30 within the slots 27, the cover 23 is locked into the first position, since it cannot slide within the support 22.

The cover 23 also comprises a passageway 32 in the top long side of the body 28. The passageway 32 is positioned in the centre of the top long side and opens onto the hollow inside of the body 28.

The strip (not shown) is held within the support 22. The sample pad is aligned with the opening 25, and sits within the body 28 of the cover 23. In the first position the body 28 sits between the opening 25 and the sample pad, preventing a sample from being deposited. In the second position the passageway 32 of the cover 32 is aligned with the opening 25 and the sample pad, such that the sample pad is exposed through the opening 25 and a sample can be deposited.

A reader (not shown) for the second embodiment of the test 21 will be similar to the reader 16 of the first embodiment of the test 1. The difference will be that instead of having the projection 19 of the first embodiment, it will have two retractable projections situated nearby the opening of the reader. The projections are biased by a biasing means in the from a of respective springs towards being extended. The tips of the projections are also shaped to present a curved surface to the test 21 when it is inserted into the reader, and an opposite curved surface. These curved surfaces mean the projections are pushed out of the way of the test 21 when it is inserted or removed from the reader, retracting.

When the test 21 is fully inserted each projection will face a respective foot 30. This biasing of the projections forces them into the slots 27 and the feet 30 out of the slots 27, since the force applied by the biasing means of the projections overcomes the resistance of the legs 29 to being bent and so forces them to bend. The projections and slots 27 interlock, and the feet 30 are displaced out of the slots 27 and further within the support 22. With the feet 30 forced out of the slots 27, the cover can now be slid from the first position to the second position by a user pushing the first end of the body 28 into the support 22.

The embodiment of the lateral flow test 44 shown in FIGS. 11 and 12 has all the same features as the embodiment of the lateral flow test 1 shown in FIGS. 1 to 6, along with the following additional features. The support 3 has a catch 40 projecting from it into the internal space of the lateral flow test, and the cover 2 has a corresponding catch 41 projecting into the internal space as well. The catches 40, 41 are positioned opposite each other when the lateral flow test 44 is in the first position. Each catch 40, 41 takes the form of a saw tooth, the gentle slopes of each saw tooth being angled in opposite directions along the length of the lateral flow test 44. The gentle slope of the support catch 40 extends from the support 3 to the top of the catch 40, the horizontal component of the extension being away from the opening 5. The gentle slope of the cover catch 41 extends from the cover 2 to the top of the catch 41, the horizontal component of the extension being towards the opening 5. Each catch 40, 41 extends into the internal space far enough such that the top parts of the catches 40, 41 overlap. The steep slopes of the catches 40, 41 are perpendicular to the surfaces of the support 3 and cover 2 from which they extend.

When the lateral flow test 44 is inserted into the reader 16 and moved into its second position, the catches 40, 41 slide past each other along their gentle slopes and then pass each other for the lateral flow test 44 to reach the second position. Due to the overlap and the steep slopes facing each other, the catches 40, 41 then prevent the lateral flow test 44 from moving back into the first position.

The embodiment of the lateral flow test 45 shown in FIGS. 13-16 has all the features of the embodiment shown in FIGS. 7-10, and the following additional features, a catch 43 extends from the support 22, adjacent the slots 27. The slots 27 lie between the catch 43 and the opening 25. The catch 43 is a saw tooth, the gentle slope of the saw tooth extending from the support 22 to the top of the catch 43, the horizontal component of the extension being away from the opening 25. The steep slope is perpendicular to the surface of the support 22 from which it extends.

The feet 30, rather than being square, are saw tooth shaped, the slopes of the saw tooth extending from the base of the feet 30 and away from the legs 29 such that they extend into the slots 27 when the feet 30 are positioned in the slots 27 when the lateral flow test 45 is in the closed position. The gentle slope extends from the base of each foot 30 to the top of the foot 30, the horizontal component of the extension being towards the opening 25. The steep slope is perpendicular to the base of the foot 30.

In use, when the lateral flow test 45 is moved to the second position the feet 30 (specifically the gentle slope of the feet 30) slide on the gentle slope of the catch 43 and then past the catch 43 into the second position. The legs 29 will then spring the feet 30 upwards such that the steep slope of the feet 30 face the steep slope of the catch 43. The steep slopes facing each other prevent the lateral flow test 45 moving back into the first position.

The one or more embodiments are described above by way of example only. Many variations are possible without departing from the scope of protection afforded by the appended claims.

1. A lateral flow test, the lateral flow test comprising:
a lateral flow assay strip comprising a sample pad arranged to receive a sample for testing; and
a cover operable to move with respect to the strip between a first position in which the sample pad is covered by the cover and a second position in which the sample pad is exposed; and
an interlocking element operable to interlock with a lateral flow test reader,
wherein the interlocking element is positioned such that the interlocking element interlocking with a lateral flow test reader enables the sample pad to be exposed.

2. A lateral flow test according to claim 1, wherein the interlocking element is arranged such that, in use, when the interlocking element is interlocked with a lateral flow test reader the cover is moves relative the strip from the first position to the second position.

3. A lateral flow test according to claim 1, wherein the cover is lockable in, and unlockable from, the first position.

4. A lateral flow test according to claim 3, wherein the interlocking element is arranged such that, in use, when the interlocking element is interlocked with a lateral flow test reader the cover is unlocked from the first position.

5. A lateral flow test according to claim 1 comprising a container for containing the lateral flow assay strip.

6. A lateral flow test according to claim 5 wherein the container is a cartridge.

7. A lateral flow test according to claim 5 wherein the strip comprises a test area comprising a test line and a control line.

8. A lateral flow test according to claim 7 wherein the container comprises a first aperture through which the test area is visible when the cover is in the second position relative to the strip.

9. A lateral flow test according to claim 8 wherein the container comprises a second aperture through which a back of the position at which test area of the lateral flow test strip is positioned is visible when the cover is in the second position relative to the strip.

10. A lateral flow test according to claim 9, wherein the second aperture is aligned with the first aperture and the lateral flow test strip sits between the first and second apertures.

11. A lateral flow test according to claim 1 wherein the cover may be operable to slide relative to the strip to move between the first and second positions.

12. A lateral flow test system comprising a lateral flow test according to claim 1 and a lateral flow test reader operable to read the results of the lateral flow test and output a reading, the reader comprising a counterpart interlocking element, the counterpart interlocking element being the counterpart to the interlocking element of the lateral flow test and being operable to interlock with the interlocking element of the lateral flow assay test.

13. A lateral flow test system according to claim 12 wherein the reader is operable to accept insertion of the test through it.

14. A lateral flow test system according to claim 13 wherein the reader comprises an opening sized and shaped to accept the lateral flow test.

15. A lateral flow test system according to claim 14 wherein the reader comprises a passageway sized and shaped to accept the lateral flow test when inserted into the reader.

16. A lateral flow test system according to claim 15 wherein the passageway extends within the lateral flow test reader.

17. A lateral flow test system according to claim 15 wherein the opening forms the entrance of the passageway.

18. A lateral flow test system according to claim 12 comprising a lateral flow test according to claim 7 wherein the lateral flow reader comprises one or more line detectors positioned and operable to read the test area of the strip when the lateral flow test is interlocked.

19. A lateral flow test system according to claim 18 wherein the or each line detector is positioned to face the first aperture when the lateral flow test is interlocked.

20. A lateral flow test system according to claim 18 wherein the or each line detector is operable to detect light transmitted from a respective position on the strip.

21. A lateral flow test system according to claim 20 wherein a first line detector is positioned to detect the light from a control line of the strip when the lateral flow test is interlocked, and a second line detector is positioned to detect light from a position on the strip adjacent the control line when the lateral flow test is interlocked.

22. A lateral flow test system according to claim 21 wherein the lateral flow test reader is operable to read the control line based on the difference between outputs of the first and second detector.

23. A lateral flow test system according to claim 20 wherein a third line detector is positioned to detect light from a test line when the lateral flow test is interlocked, and a fourth line detector is positioned to detect light from a position on the strip adjacent the test line when the lateral flow test is interlocked.

24. A lateral flow test system according to claim 23 wherein the lateral flow test reader is operable to read the test line based on the difference between outputs of the third and fourth detector.

25. A method of performing a lateral flow test, comprising the steps of:
providing a lateral flow test system according to claim 12;
interlocking the lateral flow test and the lateral flow test reader;
the lateral flow test reader automatically starting a timer for a set period once interlocking has occurred; and
the lateral flow test reader reading the test results after the set period.
